(19) United States
(12) Patent Application Publication
(10) Pub. No.: US 2012/0172681 A1
(43) Pub. Date: Jul. 5, 2012

(54) SUBJECT MONITOR

(75) Inventors: Hong Xia SUN, Beijing (CN); Yong Qiang WU, Beijing (CN); Kai Feng WANG, Beijing (CN); Peng Fei ZHU, Beijing (CN)

(73) Assignee: STMICROELECTRONICS R&D (BEIJING) CO. LTD, Beijing (CN)

(21) Appl. No.: 13/341,013

(22) Filed: Dec. 30, 2011

(30) Foreign Application Priority Data
Dec. 30, 2010 (CN) 201010625156.0

(51) Int. Cl. A61B 5/0205; A61B 5/11

(52) U.S. Cl. 600/301; 600/595

(57) ABSTRACT

An embodiment comprises includes an apparatus with a housing wearable by a subject and a first sensor operable to detect a position of the subject. An embodiment of the apparatus includes a second sensor operable to detect a body state of the subject, where the first body state may be a vital sign such as heart rate, blood pressure, body temperature or respiratory rate. The apparatus may also include a wireless module, and be operable to transmit body state data and position data to a remote device. The apparatus may include a gyroscope or an accelerometer, and may be operable to detect rotational change in the subject's position about and axis, linear acceleration of the subject along an axis, a change in position of the subject, or a rate of change in position of the subject.

### PRIORITY CLAIM

The instant application claims priority to Chinese Patent Application No. 201010625156.0, filed Dec. 30, 2010, which application is incorporated herein by reference in its entirety.

### SUMMARY

An embodiment includes an apparatus with a housing wearable by a subject, and a first sensor operable to detect a position of the subject.

Another embodiment of the apparatus also includes a second sensor operable to detect a state of the subject, where the state may include a vital sign such as heart rate, blood pressure, body temperature, or respiratory rate. The apparatus may also include a wireless module, and may be operable to transmit state data and position data to a remote device. The apparatus may include a gyroscope or an accelerometer, and may be operable to detect a position of the subject, a change in the position of the subject, and a rate of change in the position of the subject. For example, the apparatus may be operable to detect a rotational change in the subject's position about an axis or a linear acceleration of the subject along an axis.

For example, such an embodiment of the apparatus may be attached to the subject, monitor the position and vital signs of the subject, and wirelessly send position and vital-sign data to a remote device such as a computer or smart phone.

### BRIEF DESCRIPTION OF THE DRAWINGS

The present disclosure is presented by way of at least one non-limiting exemplary embodiment, illustrated in the accompanying drawings in which like references denote similar elements, and in which:

FIG. 1 is a frontal view of a human subject and of an embodiment of a body-monitor apparatus adhered to the human subject.

FIG. 2 is a block diagram of monitoring system that includes an embodiment of the body-monitor apparatus of FIG. 1 operatively coupled with a computer and a smart-phone.

FIG. 3 is diagram of the human subject of FIG. 1 and of a coordinate system for the subject's frame of reference.

FIG. 4 is a diagram of an embodiment of the body-monitor apparatus of FIGS. 1 and 2 and of a coordinate system for the apparatus' frame of reference.

FIG. 5 is a diagram of a coordinate system for a frame of reference within which the human subject of FIG. 3 and the body monitor of FIG. 4 may be located.

FIGS. 6 and 7 are side views of the human subject of FIG. 1 laying on his/her back and sitting up, respectively.

FIG. 8 is a front view of the human subject of FIG. 1 while he/she is moving from laying on his/her side to a sitting-up position.

FIGS. 9 and 10 are side views of the human subject of FIG. 1 sitting in a wheelchair and standing up from the wheelchair, respectively.

### DETAILED DESCRIPTION

A subject, such as a medical patient, may require monitoring of his/her vital signs so that doctors may diagnose and treat a disease or other affliction of the subject. For example, vital signs such as heart rate, blood pressure, body temperature, and respiratory rate may be monitored by attaching various sensors to a patient.

Unfortunately, monitoring patient vital signs may require that the patient have a plurality of sensors attached to him/her, with the sensors being attached to the monitoring devices via a plurality of wires. Such monitoring may be uncomfortable for a patient, and may require that he/she remain in bed, or at least stay within close proximity of monitoring devices to which he/she is connected.

In addition, a conventional monitoring device may be unable to detect patient movement, and, therefore, may be unable to allow one to attribute changes in patient vital signs to patient movement. For example, a conventional monitoring device may be unable to allow one who is monitoring vital signs remotely to attribute a sudden increase in heart rate to the patient moving from a supine position to a sitting position.

Moreover, such a monitor may be unable to provide an alert when a patient moves in a way that may negatively affect the patient's vital signs, and thus may negatively affect the patient's well being.

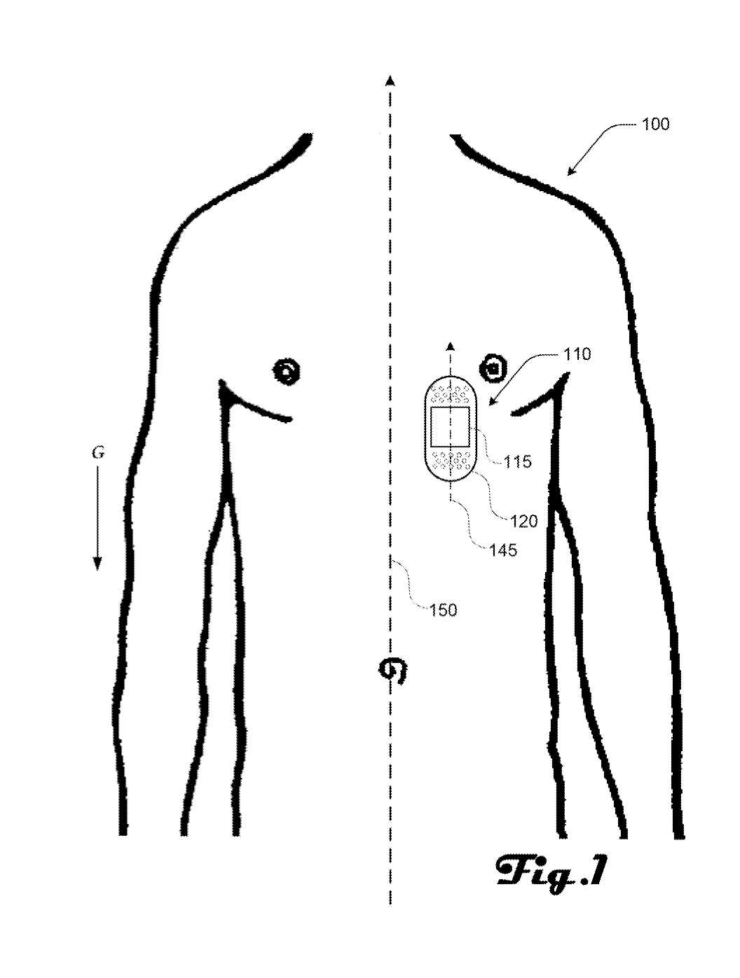

FIG. 1 is a frontal view of a human subject 100, such as a medical patient, who is “wearing” an embodiment of a body monitor 110 that is adhered to him/her with, for example, an adhesive similar to that used to attached monitor leads to a subject. As discussed in further detail herein, the body monitor 110 may comprise a monitor device 115 and an adhesive pad 120. Furthermore, the body monitor 110 may have a device axis 145, and the subject 100 may have a subject axis 150.

Compared to a conventional body monitor as discussed above, an embodiment of the body monitor 110 may provide for comfortable monitoring of one or more of the subject's 100 vital signs because the body monitor may operate wirelessly, and thereby allow the subject 100 to move about freely without being constricted by wires, without the necessity of remaining close to monitoring devices, and without the concern of dislodging monitoring sensors by pulling on sensor wires. Consequently, the monitor 110 may significantly reduce discomfort and inconvenience experienced by the subject 100 during medical observation or treatment.

In an embodiment, the body monitor 110 may be adhered to the subject 100 via the adhesive pad 120, with the device axis 145 being oriented substantially parallel to the subject axis 150. As discussed in more detail herein, approximately parallel orientation of the device and subject axes 145 and 150 allows for an assumption that the device axis is representative of the subject axis, and, therefore, that movement or other position change relative to the device axis 145 is representative of movement or other position change relative to the subject axis. Although the subject axis 150 is described as being approximately parallel to the spine (not shown in FIG. 1) of the subject 100, the subject axis 150 may have any orientation relative to the subject.

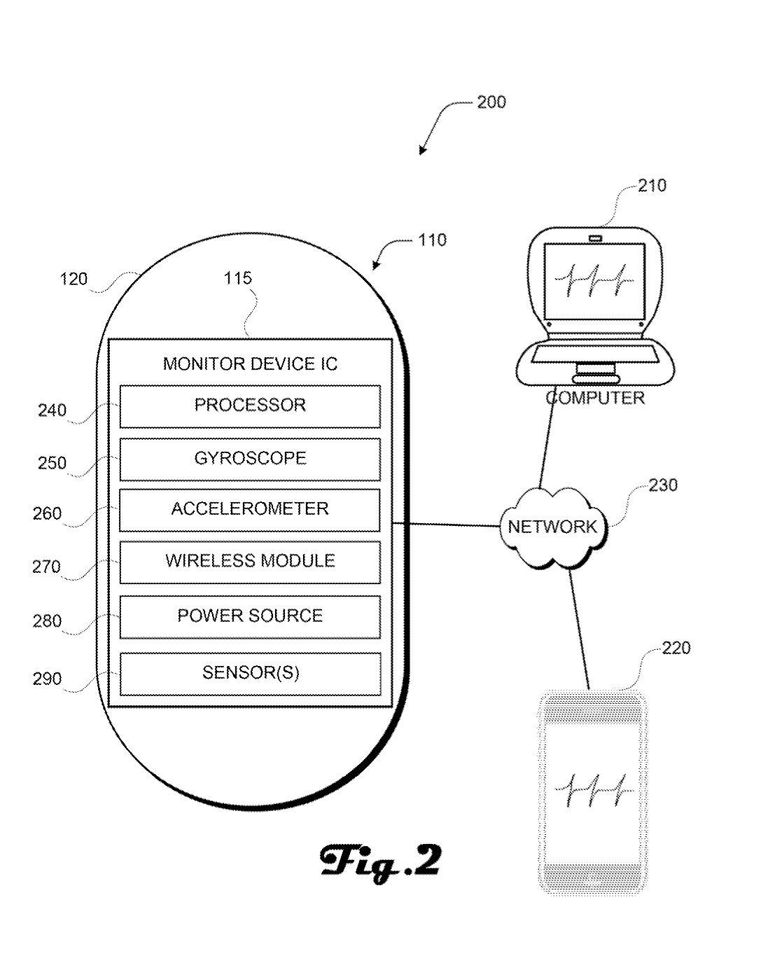

FIG. 2 is a block diagram of an embodiment of a body-monitor system 200, which includes an embodiment of the body monitor 110 of FIG. 1 operatively coupled to a computer 210 and a smart-phone 220 via a network 230.

Disposed on the adhesive pad 120 of the body monitor 110 is a monitor-device integrated circuit (IC) 115, which may be formed from one or more integrated-circuit dies. For example, the monitor device IC 115 may be a system on a chip.

The monitor-device chip 115 includes a processor 240, a gyroscope 250, an accelerometer 260, a wireless transceiver module 270, a power source 280, and one or more sensors 290.

The adhesive pad 120 may include any suitable adhesive, may be formed of any suitable material, and may be any suitable size and shape. For example, in an embodiment, the adhesive pad 120 may be similar to an adhesive bandage (e.g., a BandAid® brand adhesive bandage). The adhesive pad 120 may be constructed to allow one to adhere the body monitor 110 to the subject 100 (FIG. 1) and to remove the body monitor from the subject 100 multiple times.

In addition, the adhesive pad 120 and other components of the body monitor 110 may be made of environmentally friendly material so that if the body monitor is intended to be disposable (i.e., not reused or otherwise recovered after being removed from a subject such as the subject 100 of FIG. 1), the body monitor would have little or no negative environmental impact as waste. In a related embodiment, the adhesive pad 120 may comprise a pocket wherein the monitor-device IC 115 may be held, such that one may dispose of or recycle the pad 120 and reuse the IC 115 with a new pad 120.

The monitor-device IC 115 may be an integrated circuit, a hybrid integrated circuit, a micro-electro-mechanical system (MEMS), or any other suitable circuit or system. Furthermore, as discussed above, the components of the monitor-device IC 115 may be disposed on a single IC die or on multiple IC dies. Additionally, the monitor-device IC 115 may include more or fewer components than are described herein, and such components may be configured in any suitable arrangement.

The processor 240 may be any suitable processor, processing system, controller, or module, and may be programmable to control one or more of the other components of the body monitor 110. Furthermore, the processor 240 may perform data processing on data generated by the gyroscope 250, accelerometer 260, or the one or more sensors 290 as described in further detail herein.

The gyroscope 250 may be any suitable device operable to indicate a degree of rotation about one or more coordinate axes of the gyroscope's frame of reference. For example, the gyroscope 250 may be operable to detect “yaw”, “pitch”, and “roll” (i.e., rotation) about coordinate X, Y, and Z axes, respectively. Examples of gyroscopes suitable for the gyroscope 250 include the STMicroelectronics L3G4200DH and the L3G4200D. The gyroscope 250 may be operable to detect a change in the position, and a rate of change in position, of the body monitor 110, or of a subject that is wearing the body monitor. Alternatively, the gyroscope 250 may be operable to generate data from which a change in a subject's position, and the rates of this position change, may be calculated.

The accelerometer 260 may be any suitable device operable to indicate a linear acceleration along one or more coordinate axes of the accelerometer's frame of reference. Examples of accelerometers suitable for the accelerometer 260 include the STMicroelectronics AN2041, AN2335, or AN2381. The accelerometer 260 may be operable to detect a change in position, and a rate of change in position, of the body monitor 110, or of a subject that is wearing the body monitor. Alternatively, the accelerometer 260 may be operable to generate data from which a change in a subject's position, and the rate of this position change, may be calculated.

In an embodiment, the accelerometer 260 and gyroscope 250 may be disposed on a single die that is separate from one or more other dies of the IC 115.

The wireless module 270 may be any suitable device that is operable to send and receive wireless communications. For example, the wireless module 270 may be operable to send to the computer 210 or the smart-phone 220 data generated by the gyroscope 250, accelerometer 260, or the one or more sensors 290. Furthermore, the wireless module 270 may allow one to control the operation of one or more components of the body monitor 110, and may allow one to program the processor 240. Moreover, the wireless module 270 may send status information to the computer or smart-phone 210, 220 such as the level of power remaining in the power source 280, or the operability of the one or more sensors 290.

The power source 480 may be any suitable source of power such as a battery, and may provide power to one or more components of the body monitor 110. The power source 480 may be recharged via a wired technique, may be recharged wirelessly (e.g., via RF energy), or may be replaceable. In an embodiment, there may be a plurality of power sources 480.

The one or more sensors 290 may be operable to detect the vital signs or other body conditions of the subject 100. For example, the one or more sensors 290 may detect vital signs such as heart rate, blood pressure, body temperature, or respiratory rate. One or more of the sensors 290 may make direct contact with the skin of the subject 100, and, therefore, these sensors may extend through the adhesive pad 120 so as to contact the subject directly. The sensors 290 may be positioned in a suitable arrangement to detect one or more vital signs of the subject 100. Furthermore, in an embodiment, one or more of the sensors 290 may not be a part of the monitor-device IC 115, but may instead be operatively coupled to the monitor-device IC wirelessly or via wires. For example, where sensors 290 are positioned on different parts of a subject's 100 body to detect a vital sign or other body state, such sensors may be physically separate from the monitor-device IC 115, but may be operatively coupled to the monitor-device IC via the wireless module 270 or via wires (not shown).

The computer 210 may be any suitable computing device (e.g., a laptop or desktop computer) that is wirelessly coupled with body monitor 110, and may be operable to program the body monitor, obtain stored data from the body monitor, process data obtained from the body monitor, and the like. The computer 210 may also be operable to program the processor 240 of the body monitor 110. The computer 210 may also be operable to receive data and other related information from the body monitor 110, at a location remote from the body monitor. Accordingly, the subject 100 (FIG. 1) may be able to go about his/her normal activities such as moving, resting, or sleeping as the body monitor 110 detects one or more vital signs or body states. Data from the body monitor 110 may be sent in real time to a doctor's office or to a hospital observation station over a network 230 such as the Internet. The smart phone 220 may be operable to perform one or more functions as described above for the computer 210. Moreover, the system 200 may include one or both of the computer 210 and the smart phone 220.

In an embodiment, the computer 210 (or smart-phone 220) may provide to the subject 100, another person with the subject, or another person remote from the subject (e.g., a nurse at a monitoring station of a hospital) with an alarm or other alert relating to the vital signs or body position of the subject. For example, if the subject's 100 vital signs indicate a sudden, potentially dangerous, increase in the subject's heart rate, the computer 210 or phone 220 may provide a visual or audio alert so that the subject or a doctor may be alerted to this potentially dangerous condition. But the computer 210 or phone 220 may also provide an indication as to whether the subject 100 moved at or around the same time as the increase in heart rate, and one may use this information to determine whether the condition is dangerous. For example, if the computer 210 indicates that the subject 100 moving from a supine to sitting position coincides with the increase in heart rate, then a doctor may determine that the increase in heart rate is due to the change in position, and is not dangerous. Furthermore, in some instances (as discussed in more detail herein), movement of the subject 100 may be the cause of a dangerous body condition, and the computer 210 or smart phone 220 may alert the subject to cease such movement, and to refrain from such movement in the future to prevent a recurrence of the condition. Alternatively, an alerted doctor may be able to instruct the subject 100 to cease or refrain from such movement that causes a dangerous condition.

In an embodiment, the body monitor system 200 may also be used to capture data relating to a non-human subject 100. In addition, the body-monitor system 200, or components thereof, may be used to capture data relating to the position, movement, or condition of non-living systems, such as machinery, a vehicle, a computing device, or the like.

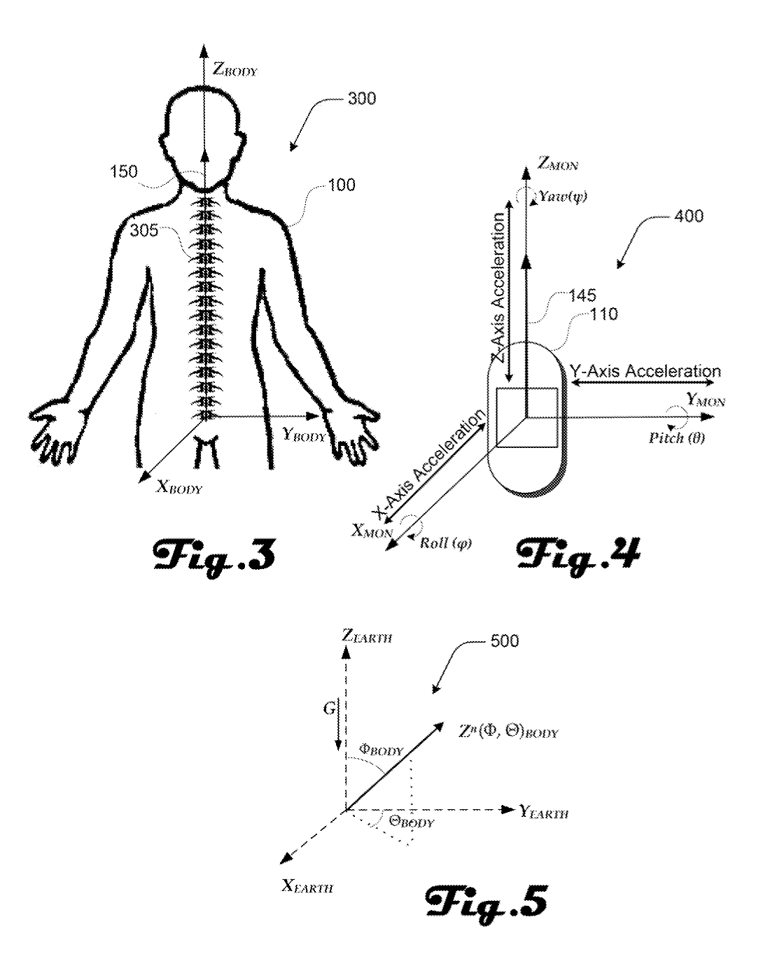

FIG. 3 is a coordinate system 300 of a frame of reference of the subject 100, the coordinate system having the axes X_{BODY}, Y_{BODY}, and Z_{BODY} interposed on the subject, where, in an embodiment, the Z_{BODY} axis is aligned with a body axis 150 of the subject. Given that the spine 305 of the subject 100 is not typically linear within the coronal plane of the subject, the Z_{BODY} axis and the body axis 150 may be aligned with a hypothetically straightened spine, or may be aligned with the spine only along the sagittal plane. As the subject 100 changes position, X_{BODY}, Y_{BODY}, and Z_{BODY} remain stationary relative to the subject. In other words, X_{BODY}, Y_{BODY}, and Z_{BODY} are fixed relative to the subject's 100 frame of reference. For example, if the subject 100 lies down (FIGS. 6-8), then the Z_{BODY} axis will maintain the same alignment with the body axis 150, even though both of these axes will move relative to the earth's frame of reference (discussed below).

In an embodiment as depicted in FIG. 3, the X_{BODY} axis extends along the mid-sagittal plane of the subject 100 perpendicular to the frontal plane of the subject, and the Y_{BODY} axis is perpendicular to the mid-sagittal plane of the subject, and is co-linear with and along the frontal plane of the subject. The Z_{BODY} axis extends in alignment with the body axis 150 (i.e., parallel to the body axis superiorly from the axis origin). Although only the positive portions of the X_{BODY}, Y_{BODY}, and Z_{BODY} axes are shown in FIG. 3, it is understood that these axes also have respective negative portions.

In an embodiment, the body monitor axis 145 (FIGS. 1-2) of the body monitor 110 is assumed to represent, i.e., be aligned with, the Z_{BODY} axis and the body axis 150. But because the body monitor 110 may be worn on the outside of the subject 100, the Z_{BODY} axis and the body monitor axis 145 may not be directly aligned. Therefore, an assumption may be made that the body monitor axis 145 is aligned with the Z_{BODY} axis, and thus that the body monitor 110 frame of reference is the same as the subject 100 frame of reference 300. Accordingly, the body monitor 110 worn by the subject 100 may be assumed to be detecting changes in the orientation of the subject frame of reference 300 relative to the earth's frame of reference as discussed further below. Alternatively, if more precise calculations are desired, then the subject frame of reference 300 may be shifted relative to the body of the subject 100 such that the Z_{BODY} axis is aligned with the body-monitor axis 145.

Although the X_{BODY}, Y_{BODY}, and Z_{BODY} are depicted as having specific orientations relative to the body of the subject 100, in another embodiment, the X_{BODY}, Y_{BODY}, and Z_{BODY} axes may have different orientations relative to the subject, and need not be aligned with a plane, the spine 305, or other part of the body. Therefore, the alignments of the X_{BODY}, Y_{BODY}, and Z_{BODY} axes shown in FIG. 3 merely represent one possible configuration of the axes.

FIG. 4 is a coordinate system 400 for the frame of reference of the body monitor 110. The coordinate system 400 has the axes X_{MON}, Y_{MON}, and Z_{MON} interposed on the body monitor 110, and are aligned (i.e., parallel or co-linear) with the respective X, Y, and Z axes of the gyroscope 250 and the accelerometer 260 (FIG. 2). As the body monitor 110 changes position, the X_{MON}, Y_{MON}, and Z_{MON} axes remain fixed relative to the body monitor. In other words, the X_{MON}, Y_{MON}, and Z_{MON} are fixed relative to the body monitor's frame of reference 400. In addition, the X_{MON}, Y_{MON}, and Z_{MON} axes may have any desired orientation relative to the frame of reference 400, the gyroscope 250, and the accelerometer 260; for example, the Z_{MON} axis need not be aligned with the body-monitor axis 145, although such alignment may make easier the calculations for determining the orientation of the body-monitor axis 145 relative to the subject's frame of reference 300 as discussed below.

Referring to FIGS. 3 and 4, in an embodiment, the Z_{BODY} axis may be aligned with, or at least considered to be aligned with, the axis Z_{MON}, and the axes X_{BODY} and Y_{BODY} may be aligned with, considered to be aligned with, or considered to be parallel to, the axes X_{MON} and Y_{MON}, respectively.

FIG. 5 is a terrestrial coordinate system 500 having the axes X_{EARTH}, Y_{EARTH}, and Z_{EARTH}, wherein the Z_{EARTH} axis is aligned with vector \vec{G}, which represents the magnitude and direction of the gravitational force of the earth. Depicted within the coordinate system 500 is a body orientation Z^{t}_{BODY}. The terrestrial coordinate system 500 is fixed to the earth's frame of reference. Additionally, as discussed above in conjunction with FIGS. 3-4, the body orientation Z^{t}_{BODY}, represents the orientation of the subject's 100 frame, and also the orientation of the body monitor's 110 frame of reference, relative to the origin of terrestrial coordinate system 500 at a time ‘t’—to simplify at least some analyses, one may assume that the origins of the coordinate systems 300 and 500 are coincident.

The Z^{t}_{BODY} orientation represents an orientation of the Z_{BODY} axis (FIG. 3) relative to the terrestrial coordinate system 500 at a given time N, e.g., Z¹_{BODY}, Z²_{BODY}, Z³_{BODY}, etc. For example, as the subject 100 changes position (e.g., lies down, bends over, reclines, etc.) the orientation of the Z_{BODY} axis of the subject 100 would change relative to the terrestrial coordinate system 500.

Z^{t}_{BODY} may be defined within the terrestrial coordinate system 500 by spherical coordinates relative to the earth X_{EARTH}Y_{EARTH}Z_{EARTH} coordinate system 500. For example, Θ_{BODY} and Φ_{BODY} are depicted in FIG. 5 as spherical coordinates of Z^{t}_{BODY}, where Θ_{BODY} represents an angle from the positive Y_{EARTH} axis projected in the X_{EARTH}Y_{EARTH} plane (e.g., in radians from 0 to 2π) with the vertex being the origin, and where Φ_{BODY} represents an angle from the positive Z_{EARTH} axis (e.g., in radians from 0 to π) with the vertex being the origin. Accordingly, Θ_{BODY} and Θ_{BODY}, for example, define the orientation/direction of Z^{t}_{BODY} from the origin of the X_{EARTH}Y_{EARTH}Z_{EARTH} coordinate system 500.

A doctor, for example, may initially calibrate the body monitor 110 by having the subject 100 stand while wearing the body monitor such that Z_{MON} is coincident with the gravitational force of earth \vec{G}, and is parallel to Z_{BODY}, as depicted in FIG. 1; this is the initial or home position. The body monitor 110 may be calibrated or synchronized to recognize the home position, e.g., by pressing a button on the computer 210 or the smart phone 220 (FIG. 2), or on the monitor 110 itself. The processor 240, computer 210, or smart phone 220 may thereafter track the orientation of the body monitor 110 relative to the terrestrial coordinate system 500 in relation to this home orientation.

As Z^{t}_{BODY} changes position relative to the terrestrial coordinate system 500 as the subject 100 moves and changes position, knowing the orientation of Z^{t}_{BODY}, or the change in the Z_{BODY} orientation relative to the X_{EARTH}Y_{EARTH}Z_{EARTH} coordinate system 500, may aid in the interpretation of body condition data detected by the body monitor 110. For example, referring to FIGS. 6-8, for a given heart rate detected by the body monitor, it may be important to determine whether the subject 100 is laying-down, sitting-up, or in the process of moving from a laying-down position to a sitting-up position, or the rate at which the subject is changing position. Detecting an elevated heart-rate while the subject 100 is moving from a laying-down position to a sitting-up position may only be indicative of exertion during the movement, and may not be cause for concern; however, an elevated heart-rate while the subject is motionless may be indicative of heart distress and require intervention by medical staff.

Also, for example, where it is dangerous for a subject's 100 heart-rate to be above a defined threshold, a rising heart rate due to exertion while the subject is moving can trigger an alert for the subject to cease movement or to slow the rate of movement. The subject 100 may, therefore, prevent dangerous body conditions (e.g., a heart rate that is too high) by restricting or modifying movement based on feedback provided by the body monitor system 200 (FIG. 2), without the necessity for intervention by medical staff.

However, should the subject 100 fail, or be unable, to control or prevent the occurrence of undesired body conditions, medical staff may be alerted by the body monitor system 200 (FIG. 2), and medical attention or instructions may be provided to the subject to prevent or treat an undesired body condition. Accordingly, the body monitor system 200 may be operable to provide different alerts to different devices based on the device user. For example, if a doctor is the user of the computer 210 and the subject 100 is the user of the smart-phone 220, the smart-phone may provide substantially more alerts to the subject 100, or different types of alerts, compared to alerts being provided to the doctor via the computer 210. The subject 100 may receive alerts via the smart-phone 220 only when the undesirable body conditions may be correctable by a change in patient behavior, whereas the doctor may only receive alerts when the subject is unable to prevent or correct undesirable body conditions, or when the subject fails to modify his or her behavior to prevent or correct undesirable body conditions. Therefore, a doctor or other medical staff may receive personalized alerts only when intervention is potentially warranted, and the subject 100 may receive personalized alerts only when the subject (or a nearby attendant) may personally intervene to prevent or correct an undesirable body condition; alternatively, the doctor, the subject, or both the doctor and the subject may receive all alerts. An alert may be triggered based on various alert criteria, and an alert may include an audio alert, a visual alert, a vibratory alert, an e-mail, an SMS text message, or the like.

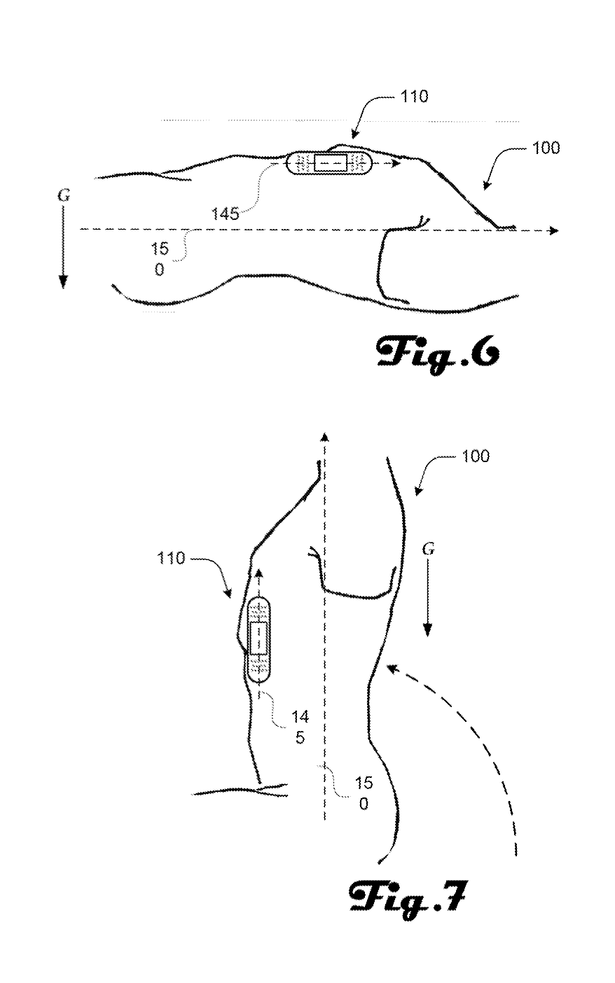

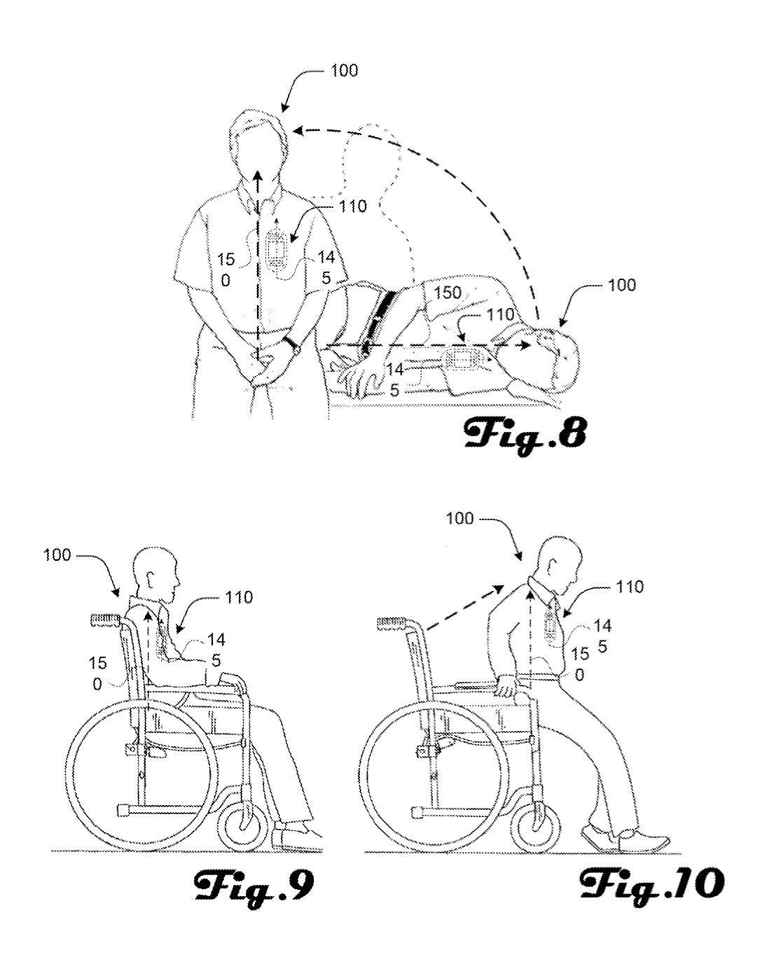

FIG. 6 depicts the subject 100 at rest in a supine position, FIG. 7 depicts the subject moving from a supine position to a sitting-up position, and FIG. 8 depicts the subject 100 moving sideways from a laying-down-on-his-side position to a sitting-up position.

FIGS. 6 and 7 depict an instance where the orientation of the subject 100 changes approximately about a single axis. In the illustrated embodiment, the subject is shown changing position approximately about the Y_{BODY} axis (which is in a direction normal to the page of FIGS. 6 and 7) when the subject alternates between the positions shown in FIGS. 6 and 7. For example, such a change in position may occur when the subject 100 sits up in bed, or when the subject lays down in bed. In such an instance, the gyroscope 250 may detect a ±90° change in pitch, i.e., a ±90° rotation about the Y_{MON} axis. Assuming that the axes Y_{BODY} and Y_{MON} may be considered to be aligned, this ±90° rotation detected by the body monitor 110 may be assumed to be indicative of and represent a ±90° rotation of the subject 100 about the Y_{BODY} axis. Therefore, from information provided by the gyroscope 250, the processor 240, computer 210, or phone 220 may determine a position of the subject 100 relative to the earth coordinate system 500 before, after, or both before and after the movement. For example, the processor 240, computer 210, or phone 220 may determine whether the subject 100 is sitting up or laying down. Furthermore, the processor 240, the computer 210, or the smart phone 220 may determine the rate or the acceleration at which the subject 100 rotated. If the processor 240, computer 210, or phone 220 detects, for example, a concurrent rise in heart rate, then the processor, computer, or phone may determine that the rise in heart rate is due to the movement of the subject, and not due to a serious medical condition. And if the detected heart rate exceeds a threshold, the processor, computer, or phone may issue an alert to the subject 100 that his/her movement was such that it caused an undesirable increase in heart rate.

In a similar manner, as the subject 100 changes position as shown in FIG. 8, the gyroscope 250 may detect a rotation about an axis that is different than the axis of rotation detected due to the position change discussed above in conjunction with FIGS. 6 and 7. The subject 100 is shown changing position approximately about the X_{BODY} axis when the subject alternates between the positions shown in FIG. 8. In such an instance, the gyroscope 250 may detect a ±90° change in roll, i.e., a ±90° rotation about the X_{MON} axis, and this rotation detected by the body monitor 110 may be assumed to be indicative of and represent a ±90° rotation of the subject 100 about the X_{BODY} axis. And the processor 240, the computer 210, or the smart phone 220 may determine the position of the subject 100, and the rate or the acceleration at which the subject rotated, as discussed above in conjunction with FIGS. 6-7.

While examples of movements that may result in a rotation about a single X, Y, or Z coordinate axis are discussed above in conjunction with FIGS. 6-8, the subject 100 may move in a manner that would result in a rotation about an axis that is displaced linearly or angularly from the X,Y, and Z coordinate axes. Accordingly, it may be desirable to calculate the rotation of the subject 100 about such a displaced axis. Such rotation may be calculated from the vector sum of the component rotations (pitch, yaw, and roll) about the X_{MON}Y_{MON}Z_{MON} axes via known methods.

FIGS. 9 and 10 depict the subject 100 changing position in a manner where the subject moves in such a way that at least his/her torso not rotate significantly about any of the coordinate axes X_{BODY}, Y_{BODY}, or Z_{BODY}, or about any other axes. For example, as the subject 100 sits down or stands up in a wheelchair, he/she moves approximately linearly along the subject axis 150 and the body-monitor axis 145 within the earth frame of reference 500 (FIG. 5), but may not rotate appreciably about any axis. Therefore, the gyroscope 250 (FIG. 2) may be ineffective in detecting this movement, because the subject 100 exhibits little or no roll, pitch, or yaw for the gyroscope to detect. But an accelerometer, such as the accelerometer 260, having a measurement axis approximately aligned with the Z_{BODY} axis may detect such a linear movement. For example, from information provided by the accelerometer 260, the processor 240, computer 210, or phone 220 may determine a position of the subject 100 relative to the earth coordinate system 500 before, after, or both before and after the movement. For example, the processor 240, computer 210, or phone 220 may determine whether the subject 100 is sitting or standing. Furthermore, the processor 240, computer 210, or phone 220 may detect the rate or acceleration of a subject 100 as he/she moves, e.g., sits down or stands up. If the processor 240, computer 210, or phone 220 detects, for example, a concurrent rise in heart rate, then the processor, computer, or phone may determine that the rise in heart rate is due to the movement of the subject, and not due to a serious medical condition. And if the detected heart rate exceeds a threshold, the processor, computer, or phone may issue an alert to the subject 100 that his/her movement was such that it caused an undesirable increase in heart rate.

Moreover, the accelerometer 260 may have more than one measurement axis, and may have an axis aligned other than with the Z_{BODY} axis to detect, for example, sudden side-to-side movements such as the subject 100 may experience in a car accident.

Accordingly, in an embodiment, changes in a position of the subject 100 and movement of the subject 100 may be detected by both the gyroscope 250 and accelerometer 260 (FIG. 2), and these changes may be collectively used to detect all types of movement of the subject 100, the rates or accelerations of such movements, and to generate warnings or alerts related to undesirable body states detected by the sensors 290 (FIG. 2).

While FIGS. 9 and 10 depict movement that results in detected acceleration substantially along one axis, the subject 100 may also move in a manner that results in detected acceleration along a plurality of axes. Accordingly, movement or change in position may be indicated as the component of acceleration about a plurality of axes. Additionally, the outputs of both the gyroscope 250 and accelerometer 260 may be used simultaneously to detect complex movement. Also, the accelerometer may have other axes that may be assumed to be aligned with the X_{BODY}, Y_{BODY} axes.

For example, if the body monitor 110 detects in a subject 100 a dangerous rise in heart rate when the accelerometer detects substantial and varied accelerations, and the gyroscope also detects substantial rotations, over a period of time, it is likely that the subject is participating in sports or another activity which causes substantial bodily exertion, and an alert may be provided to the subject 100 that such exertion is causing a dangerous rise in heart rate, or a doctor may be alerted that the subject 100 should be instructed to not exert himself/herself in sports or in other such activities so as to prevent a dangerous rise in heart rate.

In an embodiment, an alert may be provided based on rate of change in position. For example, the subject 100 may sit-up quickly or may sit-up slowly (FIGS. 6-7), and sitting-up quickly may cause a dangerous rise in heart rate, whereas sitting-up slowly does not cause a dangerous rise in heart rate. Accordingly, an alert may be provided to the subject 100 which indicates that the subject should reduce or slow his/her rate of movement so as to prevent or stop a dangerous rise in heart rate.

In one embodiment, an alert may be provided based on absolute position. For example, the subject 100 may be in a laying-down position (FIGS. 6) or may be in a sitting-up position (FIGS. 7), and being in a sitting-up position may cause a dangerous rise in heart rate, whereas being in a laying-down position does not cause a dangerous rise in heart rate. Accordingly, an alert may be provided to the subject 100 which indicates that the subject should remain in or return to a laying-down position so as to prevent or stop a dangerous rise in heart rate.

In an embodiment the body monitor 110 may be operable to control various devices or components within a room or other area proximate to the body monitor. The body monitor 110 may be able to communicate with a home automation system, components of a room, or the like. For example, the body monitor 110 may be operable to turn lights on and off depending on whether the subject 100 is detected to be laying-down or sitting-up in bed, and turning a light on or off may be achieved via a home automation system that controls a light or via a system that only controls that light. Such changes may be triggered based on home configuration criteria or location modification criteria.

In an embodiment, the body monitor 110 may itself be a smart-phone or other computing device, which includes one or more sensors 290. In such an embodiment, the body monitor 110 may be coupled to the subject 100 via a harness, may reside within the subject's clothing, or may reside within an adhesive pad 120 that is adhered to the subject. The body monitor 110 may be operable to be partially disabled when not in proximity to, or coupled to the subject 100.

From the foregoing it will be appreciated that, although specific embodiments have been described herein for purposes of illustration, various modifications may be made without deviating from the spirit and scope of the disclosure. Furthermore, where an alternative is disclosed for a particular embodiment, this alternative may also apply to other embodiments even if not specifically stated.

1. An apparatus comprising:
a housing wearable by a subject; and,
a first sensor operable to detect a position of the subject.

2. The apparatus of claim 1, further comprising a second sensor operable to detect a first body state of the subject.

3. The apparatus of claim 2, wherein the first body state is one of heart rate, blood pressure, body temperature, and respiratory rate.

4. The apparatus of claim 2, further comprising a wireless module.

5. The apparatus of claim 4, wherein the wireless module is operable to transmit first body state data and position data to a remote device.

6. The apparatus of claim 2, further comprising a processor.

7. The apparatus of claim 6, wherein the processor is operable to associate first body state data and position data with a time.

8. The apparatus of claim 1, further comprising an adhesive portion operable to adhere the apparatus to a subject.

9. The apparatus of claim 1, further comprising a power supply.

10. The apparatus of claim 1, wherein said first sensor comprises a gyroscope.

11. The apparatus of claim 10, wherein the first sensor and the gyroscope are disposed on a single integrated circuit die.

12. The apparatus of claim 2, wherein the apparatus is operable to detect a second body state.

13. The apparatus of claim 12, wherein the apparatus is operable to detect a third body state.

14. The apparatus of claim 1, further comprising a memory.

15. The apparatus of claim 14, wherein the memory is operable to store a plurality of first body state data and a plurality of position data, each being associated with a time.

16. The apparatus of claim 1, wherein the first sensor is operable to detect rotation change in position about at least one axis.

17. The apparatus of claim 1, wherein the first sensor is operable to detect linear acceleration along at least one axis.

18. The apparatus of claim 1, wherein the first sensor is operable to detect a change in position.

19. The apparatus of claim 1, wherein the first sensor is operable to detect a rate of change in position.

20. The apparatus of claim 1, wherein the first sensor is operable to detect a position relative to an earth frame of reference.

21. The apparatus of claim 1, wherein the second sensor is an accelerometer.

22. The apparatus of claim 1, wherein the second sensor comprises an accelerometer and a gyroscope.

23. A system comprising:
a first apparatus comprising:
a housing wearable by a subject; and,
a first sensor operable to detect a position of the subject; and, a second apparatus operable to receive subject position data from the first apparatus.

24. The system of claim 23, further comprising a second sensor operable to detect a first body state of the subject.

25. The system of claim 24, wherein the second apparatus is operable to receive first body state data from the first apparatus.

26. The system of claim 25, wherein the second apparatus is operable to provide an alert when at least one of received body state data and subject position data meet defined alert criteria.

27. The system of claim 26, wherein the alert is one of an audio alert, a visual alert, an e-mail, and an SMS text message.

28. The system of claim 25, wherein the second apparatus is operable to communicate with and configure a home automation system when at least one of received body state data and subject position data meet defined home configuration criteria.

29. The system of claim 25, wherein the second apparatus is operable to turn a light on or off when at least one of received body state data and subject position data meet defined home configuration criteria.

30. The system of claim 24, wherein the first apparatus is operable to detect a second body state.

31. The system of claim 30, wherein the first apparatus is operable to detect a third body state.

32. The system of claim 23, wherein the second apparatus is one of a cellular telephone, a smart-phone and a computer.

33. The system of claim 24, wherein the second apparatus comprises a display operable to display first body state data.

34. The system of claim 23, wherein the first sensor is a gyroscope.

35. The system of claim 23, wherein the first sensor is an accelerometer.

36. The system of claim 23, wherein the first sensor is operable to detect a change in position.

37. The system of claim 23, wherein the first sensor is operable to detect a rate of change in position.

38. A method comprising:
determining a position of a subject's body; and,
providing the position to a remote device.

39. The method of claim 38, further comprising determining a first body state data.

40. The method of claim 39, further comprising:
associating the first body state data with the first time; and
associating the position with the first time.

41. The method of claim 39, further comprising:
determining if one or more of the position and first body state data meet alert criteria.

42. The method of claim 41, further comprising:
providing an alert where one or more of the position and first body state data meet alert criteria.

43. The method of claim 39, further comprising:
determining if one or more of the position and first body state data meet location modification criteria.

44. The method of claim 43, further comprising:
turning a light on or off if one or more of the position and first body state data meet location modification criteria.

45. The method of claim 39, further comprising:
determining a second body state data.

46. The method of claim 45, further comprising:
determining a third body state data.

47. The method of claim 38, wherein the position is determined via a gyroscope.

48. The method of claim 38, wherein the position is determined via an accelerometer.

49. The method of claim 38, comprising detecting a change in position.

50. The method of claim 38, comprising detecting a rate of change in position.
